(19) United States
(12) Patent Application Publication
(10) Pub. No.: US 2024/0336552 A1
(43) Pub. Date: Oct. 10, 2024

(54) COMPOUNDS

(71) Applicant: MicroVention, Inc., Aliso Viejo, CA (US)

(72) Inventors: Gloria Hincapie, Lake Forest, CA (US); Petr Vasek, Costa Mesa, CA (US); Gregory M. Cruise, Rancho Santa Margarita, CA (US)

(21) Appl. No.: 18/294,911

(22) PCT Filed: Aug. 2, 2022
(86) PCT No.: PCT/US2022/074420

§ 371 Date: Feb. 2, 2024

Related U.S. Application Data
(60) Provisional application No. 63/228,454, filed on Aug. 2, 2021.

(51) Int. Cl. C07C 69/65; A61K 51/04; C07C 69/63

(52) U.S. Cl. CPC C07C 69/65; A61K 51/0402; C07C 69/63

(57) ABSTRACT

Described are improved processes for the preparation of iodized fatty acid esters from free fatty acids via in-situ formation of hydroiodic acid using silane chemistry.

### RELATED APPLICATIONS

This application is a national stage entry under 35 U.S.C. § 371 of International Patent Application No. PCT/US2022/074420, filed Aug. 2, 2022, which claims priority of U.S. Provisional Patent Application No. 63/228,454, filed Aug. 2, 2021, the entire contents of which are incorporated herein by reference.

### FIELD

Described herein are iodinated compounds for medical imaging.

### SUMMARY

The present invention relates to an improved process for the preparation of iodized fatty acid esters from free fatty acids via in-situ formation of hydroiodic acid using silane chemistry. The iodinated liquid may be used for imaging of vascular sites and cavities within the body or used with an aqueous phase to prepare a drug loaded radiopaque embolization emulsion with optimal physical-chemical properties which can be delivered via catheter. The iodinated liquid when used as an imaging agent during a procedure such as hysterosalpingography may also improve fertility outcomes as compared to use of a water based imaging agent.

Iodine-containing compounds have many uses and many synthetic methodologies have been tried. Most iodized oils available have been derived from plant seeds and are composed of a mixture of fatty acid analogs, including saturated fatty acids. The type of plant, as well as its growth conditions, dictates the fatty acid composition and the percent availability of each type.

Iodipin is derived from sesame seed oil. Iodization of the oil is achieved using iodine monochloride in alcoholic solution. The methodology simultaneously incorporates iodine and chlorine, resulting in a chloroiodized oil. This is an undesirable characteristic as the presence of chlorine results in the facile decomposition of the oil.

Iodized oils are often used as iodine containing pharmaceuticals and the deficiencies due to contaminants resulting from the available chemistry sometimes limits their use. Advancements in the chemistry field include reacting gaseous hydriodic acid at moderate temperatures with esterified fatty acids resulting in the addition of iodine to the compound. This removes the chlorine contaminant forming more stable final products. Similarly, a modified Iodipin recipe uses aqueous hydroiodic acid. This methodology results in a purely hydroiodized analog with better iodination and more stable products.

An improved method using iodine chloride to overcome disadvantages of chloroiodized oil formation lead to the synthesis of the ethiodized oil known as Lipiodol. It is composed of the ethyl esters of iodized fatty acids from poppyseed oil. Poppy seed oil is preferred over other natural oils because of certain desirable characteristics, such as an increased iodine content in the final product. Lipiodol has approximately 40% (w/w), compared to Iodipin with ˜20%, iodine content. This is due to more poly-unsaturated sites in the fatty acids comprising poppyseed oil compared to sesame seed oil.

Lipiodol has been used as an iodinated radio-opaque agent for imaging the body. Rapeseed oil can be iodinated for use in roentgenographic imaging studies. Fatty acids or esters are iodinated by mixing iodine and mercuric chloride in organic media forming the stable iodized oils. Esterifying first and using fatty acid esters instead of the free fatty acid as starting material results in improved products with greater stability. Iodinated radio-opaque agents generated from natural oils include non-iodinated oils from saturated fatty acids, which inclusion results in a lower radio-opacity due to the lower molar amount of iodine present.

Silane chemistry has been used to make iodinated compounds, by in-situ formation of iodotrimethylsilane under anhydrous conditions. For example, chlorotrimethylsilane and sodium iodide have been used in the presence of water to iodinate rapeseed oil. The resulting compound, Brassiodol, is a less expensive version of Lipiodol that can be used to eradicate goiter, for example, in developing countries. The main differences in the chemical composition stems from the fatty acids found in rapeseed oil compared to poppyseed. The major iodine-containing component in Brassiodol is an oleic acid analog while Lipiodol is mostly a linoleic derivative.

A variety of methods for preparing iodinated compounds are established and used to synthesize numerous analogs including polyiodinated benzene derivatives for use as contrast agents and deuterated alkyl iodides formed using silane chemistry in the presence of deuterated oxide.

Ethiodized oil is used in conventional transarterial chemoembolization (cTACE) for the treatment of cancer. Currently, cTACE is considered a standard of care for hepatocellular carcinoma (HCC) in many countries. Lipiodol can be used alone or as an emulsion comprising a lipid and an aqueous phase. In many instances, delivery of the emulsion is followed by embolization with particulates or microspheres. In some cases, the aqueous phase comprises a chemotherapeutic agent allowing for targeted delivery via Lipiodol. Emulsions are prepared by mixing the lipid phase with the aqueous phase and homogenizing to the desired particles size. However, emulsions need to be stable in order to have a slow targeted drug release, otherwise the therapeutic agent will be dispersed into systemic circulation. Unfortunately, there is currently no commonly accepted method to form the desired stable emulsions despite testing of various stabilizing agents.

Described herein are procedures for the preparation of iodized fatty acid esters and formation of drug loaded “water in oil” emulsions deliverable by a catheter without process complications. The synthetic methodology allows for a simplified reaction workup and removal of undesired impurities without need of extensive purification. Resulting final purified material exhibits good purity and stability during storage. In-situ formation of hydroiodic acid can be achieved by reacting an alkaline iodide with chlorotrimethylsilane, in the presence of water. This method of in-situ formation of hydroiodic acid from alkyl-chlorosilanes generates volatile impurities which can be removed by distillation and consequently reduce the amount of undesirable side products. The resulting hydroiodic acid is then reacted with a fatty acid or fatty acid ester to obtain the iodized compound.

In some embodiments, the synthetic process can be simplified using a fully characterized fatty acid or ester as the reagent. This eliminates non-reactive impurities present in natural oil-sourced fatty acid esters, mainly saturated fatty acids, and allows utilization of precise reagent ratios in the reaction, further simplifying the purification procedure. The final iodized fatty acid ester products can exhibit sufficient radiopacity to ensure visualization via imaging techniques and adequate viscosity to allow facile delivery through a catheter.

The final iodized fatty acid ester products can further be used to form an emulsion incorporating an aqueous solution of an active pharmaceutical agent. The drug-loaded radiopaque emulsion enables targeted delivery for the treatment of certain cancers.

Embodiments described herein are directed towards the preparation of an alkyl ester of iodized fatty acids via in-situ formation of hydroiodic acid using silane chemistry.

In some embodiments, the starting material can be an unsaturated fatty acid esterified prior to conversion to the iodine analog. In other embodiments, the starting material can be a free fatty acid esterified after conversion to the iodine analog.

In one embodiment, the ester component can be a methyl, ethyl or similar moiety. In another embodiment, the ester portion can be a mixture of methyl, ethyl and/or similar moiety.

In some embodiments, one or more double bonds of the fatty acid or ester can be replaced by iodine or similar halogen.

In one embodiment, the iodinated liquid can be mixed with one or more surfactant, emulsifier, or additive to impart the desired characteristics. The additive needs to be pharmacologically acceptable and results in no adverse effects after delivery. Some additives may include but not limited to amino acids, fatty acids, lipids, surfactants, therapeutics, drugs or nanoparticles.

In one embodiment, the iodinated liquid will have no toxic impurities and may be used for imaging of vascular sites and cavities within the body, such as hypervascularized tumors or arteriovenous malformations.

In some embodiments, the iodinated fatty acid ester can be used for the embolization of hypervascularized tumors or arteriovenous malformations. The iodized compound can also be used as contrast solution during surgical procedures. In another embodiment, it can be used for making emulsions for therapeutic use. These emulsions may include stabilizing agents or nanoparticles. Overall, the iodinated fatty acid ester and/or corresponding analogs can be used as a pharmaceutical solution that can be administered to a patient as a treatment for cancers.

In some embodiments, methods of preparing iodized fatty acid esters are described. The methods can include reacting a chloroalkylsilane with an alkaline salt in the presence of a solvent to form a hydroiodic acid.

In some embodiments, the reacting is performed in-situ.

In other embodiments, the chloroalkylsilane is trimethylsilyl, triethylsilyl, tert-butyldiphenylsilyl, tert-butyldimethylsilyl and triisopropylsilyl chloride, bromide or iodide, dichlorodimethylsilane, methyltrichlorosilane, or a combination thereof. The alkaline salt can be sodium iodine, potassium iodide, lithium iodide, cesium iodide, or calcium iodide.

In some embodiments, the reacting results in volatile impurities. The volatile impurities can be removed by distillation.

In other embodiments, the methods can further include reacting the hydroiodic acid with a fatty acid, a fatty acid ester, or a combination thereof to form the iodized fatty acid esters.

The fatty acid or fatty acid ester can be unsaturated or poly-unsaturated. In some embodiments, the fatty acid is oleic acid, α-linolenic acid, linoleic acid, stearidonic acid, linolelaidic acid, palmitoleic acid, arachidonic acid, or a combination thereof. In other embodiments, the fatty acid ester is derived from oleic acid, linoleic acid, α-linolenic acid, stearidonic acid, linolelaidic acid, palmitoleic acid, arachidonic acid, or a combination thereof or the fatty acid can originate from poppyseed oil, rapeseed oil, sesame oil, rice oil, corn oil, cottonseed oil, soybean oil, sunflower oil, peanut oil, flaxseed oil, hemp oil, coconut oil, olive oil or an animal or fish source oils as chicken, cod, herring, mackerel, salmon, menhaden and sardine, or a combination thereof.

In some embodiments, the iodized fatty acid esters are methyl esters, ethyl esters, propyl esters, isopropyl esters, butyl esters, or a combination thereof.

In some embodiments, the iodized fatty acid esters described herein are iodinated alkyl oleic, α-linolenic, linoleic, stearidonic, linolelaidic, palmitoleic, arachidonic acid esters, or a combination thereof.

In some embodiments, the iodized fatty acid esters include iodinated alkyl oleic acid esters selected from one or more of:

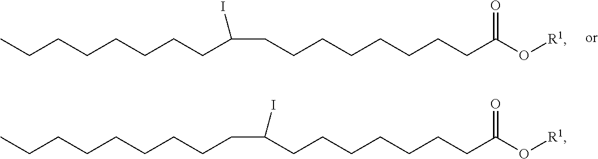

wherein
each R¹ is, independently, C₁₋₄ alkyl, for example, methyl, ethyl, propyl, isopropyl, butyl, or isobutyl.

In some embodiments, the iodized fatty acid esters include iodinated alkyl alpha linolenic acid esters selected from one or more of:

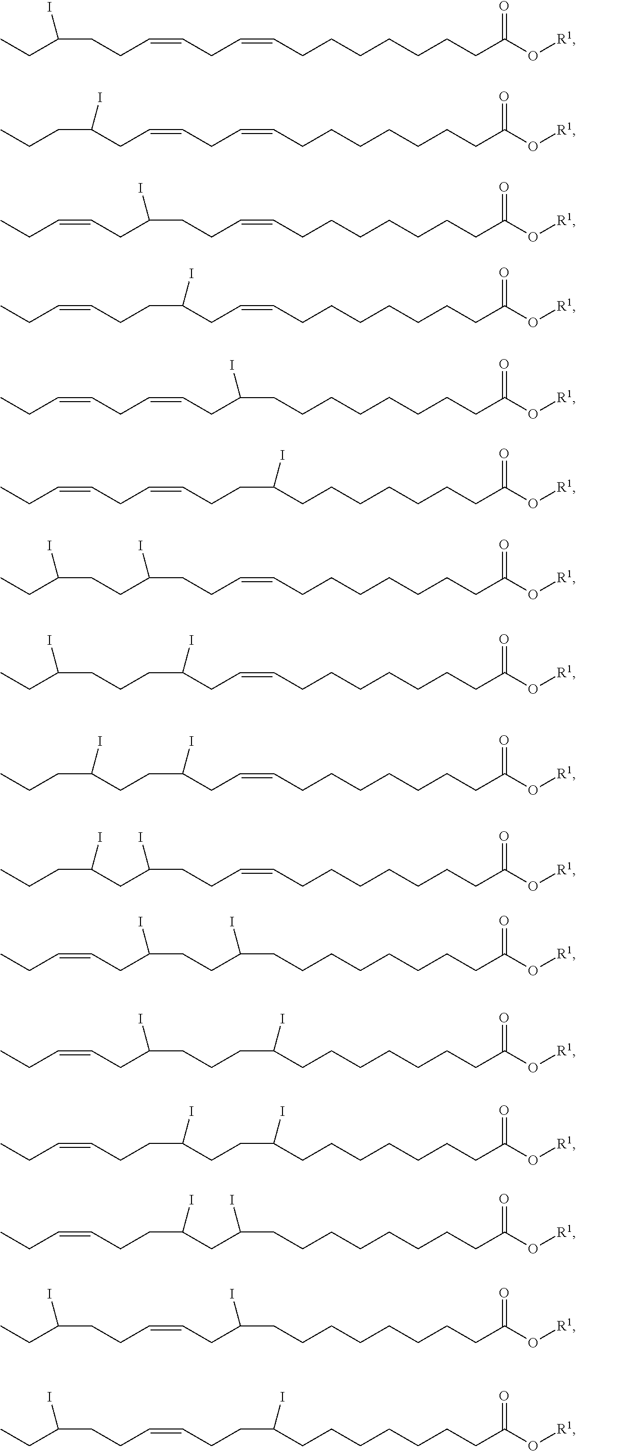

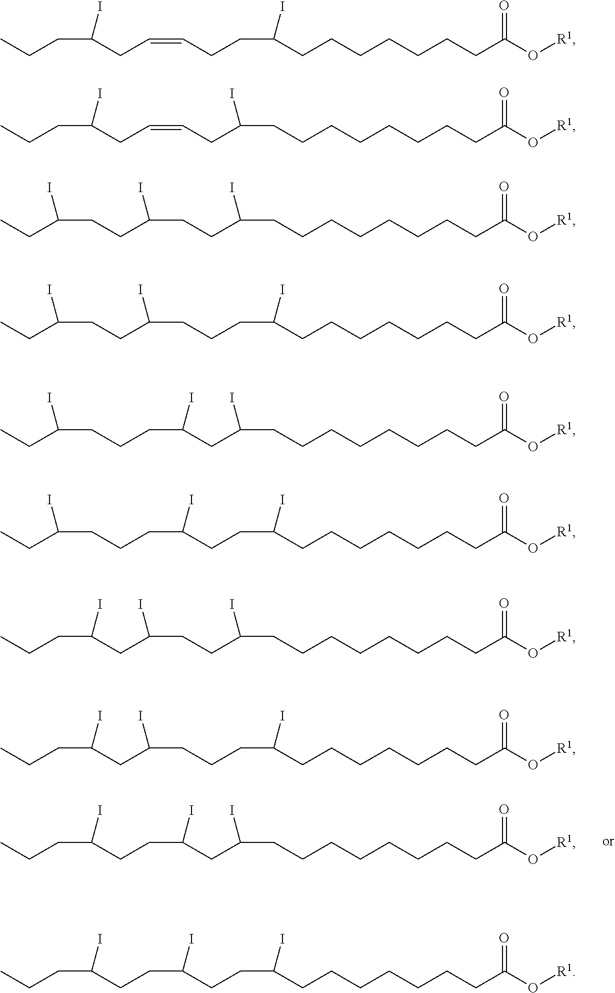

In some embodiments, the iodized fatty acid esters include iodinated alkyl linoleic acid esters selected from one or more of:

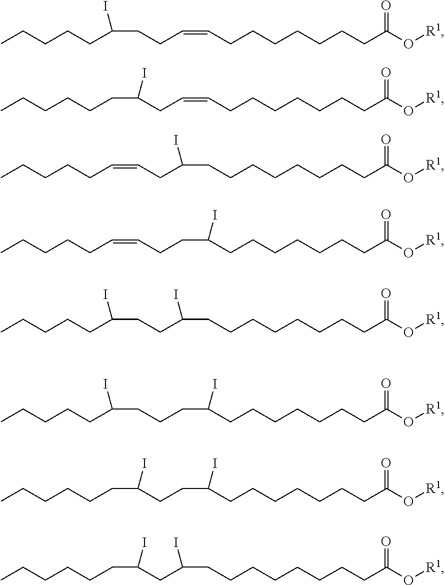

wherein
each R¹ is, independently, C₁₋₄ alkyl, for example, methyl, ethyl, propyl, isopropyl, butyl, or isobutyl.

In some embodiments, the iodized fatty acid esters include iodinated alkyl stearidonic acid esters, for example, alkyl stearidonic acid esters substituted with at least one iodine, for example, substituted with one, two, three, or four iodines, selected from one or more of:

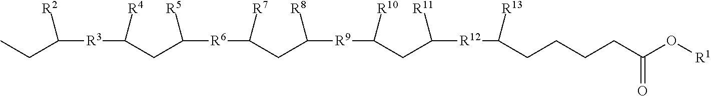

wherein
each R¹ is, independently, C₁₋₄ alkyl, for example, methyl, ethyl, propyl, isopropyl, butyl, or isobutyl;
R² is I, R³ is a single bond, and R⁴ is H, or R² is H, R³ is a single bond, and R⁴ is I, or R² is H, R³ is a double bond, and R⁴ is H;
R⁵ is I, R⁶ is a single bond, and R⁷ is H, or R⁵ is H, R⁶ is a single bond, and R⁷ is I, or R⁵ is H, R⁶ is a double bond, and R⁷ is H;
R⁸ is I, R⁹ is a single bond, and R¹⁰ is H, or R⁸ is H, R⁹ is a single bond, and R¹⁰ is I, or R⁸ is H, R⁹ is a double bond, and R¹⁰ is H; and
R¹¹ is I, R¹² is a single bond, and R¹³ is H, or R¹¹ is H, R¹² is a single bond, and R¹³ is I, or R¹¹ is H, R¹² is a double bond, and R¹³ is H.

In some embodiments, the iodized fatty acid esters include iodinated alkyl linolelaidic acid esters selected from one or more of:

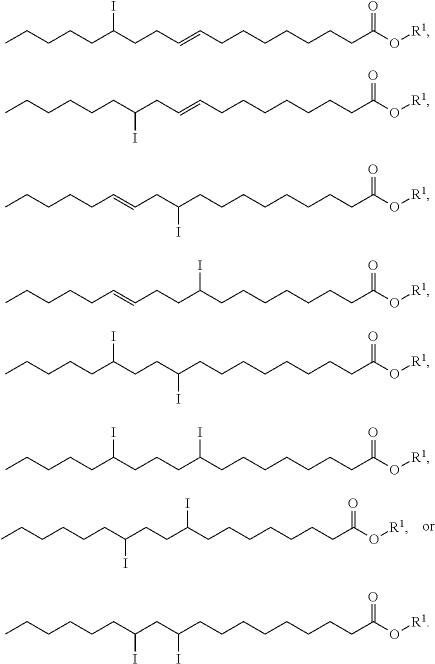

In some embodiments, the iodized fatty acid esters include iodinated alkyl palmitoleic acid esters selected from one or more of:

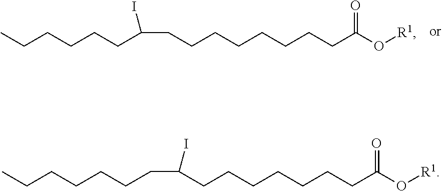

In some embodiments, the iodized fatty acid esters include iodinated alkyl arachidonic acid esters selected from one or more of:

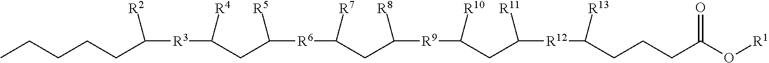

wherein
each R¹ is, independently, C₁₋₄ alkyl, for example, methyl, ethyl, propyl, isopropyl, butyl, or isobutyl;
R² is I, R³ is a single bond, and R⁴ is H, or R² is H, R³ is a single bond, and R⁴ is I, or R² is H, R³ is a double bond, and R⁴ is H;
R⁵ is I, R⁶ is a single bond, and R⁷ is H, or R⁵ is H, R⁶ is a single bond, and R⁷ is I, or R⁵ is H, R⁶ is a double bond, and R⁷ is H;
R⁸ is I, R⁹ is a single bond, and R¹⁰ is H, or R⁸ is H, R⁹ is a single bond, and R¹⁰ is I, or R⁸ is H, R⁹ is a double bond, and R¹⁰ is H; and
R¹¹ is I, R¹² is a single bond, and R¹³ is H, or R¹¹ is H, R¹² is a single bond, and R¹³ is I, or R¹¹ is H, R¹² is a double bond, and R¹³ is H.

In some embodiments, the compositions described herein include a plurality of iodized fatty acid esters, for example, one or more of the same compound or one or more of different compounds provided herein.

Natural oils include saturated fatty acids that cannot be iodinated according to the procedures described herein due to lack of an unsaturation, for example, an alkene or alkyne moiety. Lipiodol, for example, includes such saturated fatty acid moieties and thereby has a lower molar iodine content per volume than the iodinated fatty acid esters described herein. The amount of iodine content correlates to radio-opacity. Compositions described herein may include one or more iodinated fatty acid esters described herein, which may be the same iodinated fatty acid ester or a mixture of different fatty acid esters.

In some embodiments, a composition is described that comprises a plurality of the iodinated fatty acid esters described herein. In some embodiments, the moles of alkene in the plurality of the compound or compounds is equal to or less than the moles of iodine in the plurality of the compound or compounds. In some embodiments, the compositions comprise a plurality of the iodinated fatty acid esters described herein, wherein the moles of alkene in the plurality of the compound or compounds is at least 3 times less than the moles of iodine in the plurality of the compound or compounds. In some embodiments, the compositions comprise a plurality of the iodinated fatty acid esters described herein, wherein the moles of alkene in the plurality of the compound or compounds is at least 10 times less than the moles of iodine in the plurality of the compound or compounds.

In some embodiments, the reacting occurs at about 80° C. and/or the solvent is water or acetonitrile, diethyl ether, or acetone.

In some embodiments, the methods further include mixing the iodized fatty acid esters with an active pharmaceutical agent. In some embodiments, the methods further include mixing the iodized fatty acid esters with a chemotherapeutic drug.

In some embodiments, compositions are described including the iodized fatty acid esters described herein and an oil selected from vegetable oil, sesame seed oil, poppy seed oil, or a combination thereof.

Treatment methods are also described, the methods including administering a composition as described herein to a site in need thereof.

### BRIEF DESCRIPTION OF THE DRAWINGS

FIG. 1 shows the radiopacity of iodized ethyl linoleate (left loop) prepared in Example 3 as compared with lipiodol (right loop) after loading each in a separate 5 Fr. catheter.

FIG. 2 shows the HPLC analysis (ELSD) of iodized ethyl linoleate prepared in Example 3 as compared with Lipiodol.

FIG. 3 shows the HPLC analysis (UV) of iodized ethyl linoleate prepared in Example 3 as compared with Lipiodol.

FIG. 4 shows drug elution of doxorubicin from iodized ethyl linoleate as compared with Lipiodol from Example 12.

FIG. 5 shows drug elution of mitomycin C from iodized ethyl linoleate as compared with Lipiodol from Example 12.

### DETAILED DESCRIPTION

Described herein are synthetic methodologies for obtaining alkyl esters of iodized fatty acids. An iodine-containing fatty acid ester can be synthesized by mixing a trimethylchlorosilane with sodium iodide in an organic solvent system. Hydroiodic acid is formed in situ, in the presence of water, and reacts with a fatty acid or fatty acid ester of known composition.

The fatty acids used for the preparation of the iodinated analog can be characterized in that the fatty acid is selected from the group formed by unsaturated and poly-unsaturated fatty acids. Such fatty acids can include, but are not limited to oleic acid, α-linolenic acid, linoleic acid, stearidonic acid, linoleladilic acid, palmitooleic acid, arachidonic acid, or a mixture thereof, or derivatives thereof. Similarly, the fatty acid ester can be derived from oleic acid, linoleic acid, α-linolenic acid, stearidonic acid, linoleladilic acid, palmitooleic acid, arachidonic acid, or a mixture thereof, or derivatives thereof. Starting materials can also include fatty acids derived from fatty acid triglycerides containing mixture of saturated and un-saturated fatty acids originating from natural plant based oils such as poppyseed oil, rapeseed oil, sesame oil, rice oil, corn oil, cottonseed oil, soybean oil, sunflower oil, peanut oil, flaxseeds oil, hemp oil, coconut oil, olive oil or animal or fish source oils as chicken, cod, herring, mackerel, salmon, menhaden and sardine and similar, or combinations thereof, or derivatives thereof.

Iodinated analogs can be prepared from fatty acids or fatty acid esters with one unsaturated site. In another embodiment the starting material can have more than one unsaturated site. In one embodiment, after being reacted, a portion of the double bonds initially present in the fatty acid or fatty acid ester can be reacted with iodine. In another embodiment, after being reacted, all or nearly all the double bonds initially present in the fatty acid or fatty acid ester can be reacted with iodine.

The fatty acids used for the preparation of the iodinated analog can be esterified before or after iodination resulting in a final product of an iodinated fatty acid ester. The final product can be an ester of lower alkyl alcohol, including but not limited to methyl ester, ethyl ester, propyl ester, isopropyl ester, butyl ester or similar. Additionally, the iodinated fatty acid ester can also be a mixture of methyl ester, ethyl ester, propyl ester, isopropyl ester, butyl ester and/or similar esters of fatty acids.

In some embodiments, the process used for the preparation of an iodinated analog involves mixing an alkaline iodide and an alkylsilylated halide in a solvent system, followed by addition of water and the fatty acid or fatty acid ester.

The alkaline iodide can include, but is not limited to potassium iodide, sodium iodide, lithium iodide, cesium iodide and calcium iodide.

Alkylsilylated halide can be substituted alkyl and aryl silyl halide, for example, but not limited to trimethylsilyl, triethylsilyl, tert-butyldiphenylsilyl, tert-butyldimethylsilyl and triisopropylsilyl chloride, bromide or iodide, dichlorodimethylsilane, methyltrichlorosilane, mixtures thereof, and derivatives thereof.

The organic solvent system can be acetonitrile, diethyl ether, acetone, or similar organic media. Any amount of solvent can be utilized to prepare the desired iodinated analogs. Solvent concentrations can be about 10% w/w, about 20% w/w, about 30% w/w, about 40% w/w, about 50% w/w, about 60% w/w, about 70% w/w, about 80% w/w, about 90% w/w, between about 20% w/w and about 80% w/w, between about 50% w/w and about 80% w/w, or between about 30% w/w and about 60% w/w of the solution.

The reaction can be run at a temperature that produces the iodinated fatty acid analogs with desired characteristics. The reaction can be run at a temperature of about 10° C., about 20° C., about 30° C., about 40° C., about 50° C., about 60° C., about 70° C., about 80° C., about 90° C., about 100° C., or at about room temperature. In one embodiment, the reaction occurs at about 80° C. In another embodiment, the reaction is run at a temperature that is less than the boiling point of the solution.

Crude product obtained from the reaction can be worked up by simple extraction of side products or evaporation under reduced pressure. Iodine still present can be eliminated using sodium thiosulphate and filtration over basic alumina removing peroxides.

After processing, the resulting product can be a non-toxic, stable, oily liquid. It can have adequate viscosity to allow facile delivery as a medical device. The product can be radio-opaque enough for visualization via X-ray and can be used as a contrast agent during surgical procedures or imaging studies.

In some embodiments, the iodinated analog or iodized fatty acid esters may include one, two, three, or four iodines. In some embodiments, the iodized fatty acid esters do not include an unsaturation, for example, an alkene or alkyne. In some embodiments, the iodinated alkyl fatty acid esters may be selected, independently, from one or more of:

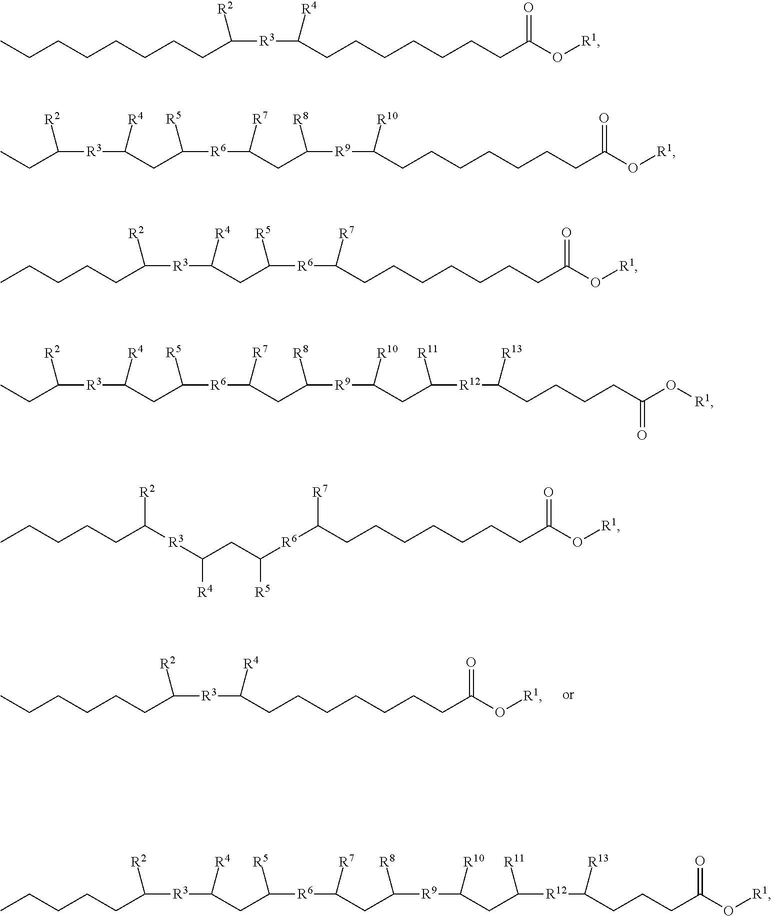

wherein
each R¹ is, independently, C₁₋₄ alkyl, for example, methyl, ethyl, propyl, isopropyl, butyl, or isobutyl;
independently, R² is I, R³ is a single bond, and R⁴ is H, or R² is H, R³ is a single bond, and R⁴ is I, or R² is H, R³ is a double bond, and R⁴ is H;
independently, R⁵ is I, R⁶ is a single bond, and R⁷ is H, or R⁵ is H, R⁶ is a single bond, and R⁷ is I, or R⁵ is H, R⁶ is a double bond, and R⁷ is H;
independently, R⁸ is I, R⁹ is a single bond, and R¹⁰ is H, or R⁸ is H, R⁹ is a single bond, and R¹⁰ is I, or R⁸ is H, R⁹ is a double bond, and R¹⁰ is H; and
independently, R¹¹ is I, R¹² is a single bond, and R¹³ is H, or R¹¹ is H, R¹² is a single bond, and R¹³ is I, or R¹¹ is H, R¹² is a double bond, and R¹³ is H.

Additionally, the iodinated analog can be used as a pharmaceutical solution including an iodinated fatty acid ester and/or corresponding derivatives that can be used as a drug. The iodinated analog can also be mixed with a chemotherapeutic drug or molecule with anti-tumor activity that can be administered for localized delivery to a patient as treatment. The resulting solution can be administered as a treatment for cancers, including the embolization of hypervascularized tumors. The resulting solution can be administered during surgical procedures, including arteriovenous malformations and occlusion of other cavities within the body.

The iodinated fatty acid ester and/or corresponding analogs can be used alone or as a pharmaceutical solution by mixing with one or more additives to form an emulsion that can be administered to a patient for therapeutic use. In one embodiment, said pharmaceutical solution comprises the iodinated fatty acid esters, an acceptable solvent and a component with anti-tumor activity. Of the pharmaceutically acceptable solvents water for injection and saline are preferred. When delivered therapeutically as an emulsion, it can be used alone or as an emulsion followed by particulate or microsphere embolization to prevent washout of the chemotherapeutic agent or to promote tissue necrosis.

The emulsion can include a lipid phase and an aqueous phase. Any ratio of lipid phase and aqueous phase can be used that allows for the desired emulsion. The lipid phase contains one or more iodinated fatty acid esters. In some embodiments, the emulsion may contain additional additives, including but not limited to, fatty acids, fatty acid esters, amino acids, alcohols, surfactants, therapeutics, or natural oils such as animal, mineral, or plant-based oils. The iodinated oil phase can include at least one vegetable oil, sesame seed oil, poppy seed oil, or mixture thereof. The lipid phase in the emulsion can be about 40% (v/v), between about 50% (v/v) to about 80% (v/v), or about 90% (v/v). The iodinated portion can be lower but should not compromise radiopacity with imaging instruments.

The aqueous phase comprises a pharmaceutically acceptable solvents such as water for injection or saline. The aqueous phase in the emulsion can be between about 20% (v/v) to about 50% (v/v). The aqueous phase may include contrast agents, stabilizing agents, viscosity changing component(s) or nanoparticles. In one embodiment, the nanoparticles will be polyester based particulates. A factor in nanoparticle selection can be the desire for degradability. Another factor in nanoparticle selection can be the desire for degradation products of the particle to elicit a negligible response from the host. In yet another embodiment, the nanoparticle can stabilize the emulsion of the lipid phase and the aqueous phase. In other embodiments, there can be desire for degradation products of the particles to elicit substantially no response from the host.

Alternative additives can include emulsifiers, lipids, drugs, anti-cancer agents, particulates, or combination thereof. In some embodiments the additive needs to be pharmacologically acceptable, with adequate stability and result in no adverse effects after delivery. They can include platinum-based antineoplastics, anticancer agents, and/or anthracycline drugs. Anticancer agents can include, but are not limited to, doxorubicin, epirubicin, idarubicin, irinotecan, cisplatin, oxaliplatin, miriplatin, streptozotocin, mitoxantrone, zinostatin stimalamer (SMANCS), mitomycin C, a combination thereof, or the like.

An emulsifying agent can be used to maintain emulsion, stabilizing the water in oil interface. Additives, including compounds to improve desirable physical-chemical properties, are usually found in about 0.1% to about 10% of the emulsion. In other embodiments, the additives can be about 15% to about 25%, from about 25% to about 75% or between 0.1% to about 90%. Additive concentration in the emulsion can be altered to obtain an emulsion with the desired characteristics.

The emulsions are prepared by mixing the aqueous phase into the lipid phase and homogenizing to a desired particle size. A total of 15 to 25 back-and-forth passes between two syringes should give the desired size. More or less mixing can be used to impart desired characteristics. After preparation, the emulsion can be delivered using a medical device such as a catheter as a treatment for patients, particularly for treatment of certain cancers.

### EXAMPLES

### Example 1: Preparation of ethyl linoleate (ethyl (9Z,12Z)-octadeca-9,12-dienoate)

Linoleic acid (50.0 g, 178.3 mmol) is dissolved in anhydrous ethanol (200 ml) and 2,2-diethoxypropane (34.5 ml; 212.9 mmol) and treated with sulfuric acid (0.3 ml; 5.6 mmol). The mixture is heated to 65° C. for a period of 16 hours and concentrated in vacuum. The mixture is re-dissolved in 250 ml of diethyl ether, extracted two times with 250 ml of saturated sodium bicarbonate aqueous solution, one time with 250 ml of water and one time with 250 ml of brine. Ether solution is dried over MgSO₄, filtered and concentrated to yield 52.1 g of product.

### Example 2: Preparation of Ethyl Oleate

Oleic acid (100.0 g, 354.0 mmol) is dissolved in anhydrous ethanol (500 ml) and treated with sulfuric acid (0.6 ml; 11.2 mmol). The mixture is heated to 65° C. for period of 16 hours and concentrated in vacuum. The mixture is re-dissolved in 500 ml of diethyl ether, extracted two times with 500 ml of saturated sodium bicarbonate aqueous solution, one time with 250 mL of water and one time with 250 ml of brine. Ether solution is dried over MgSO₄, diluted with 500 ml of hexanes, filtered through a plug of silica gel and concentrated to yield 73.2 g of product.

### Example 3: Preparation of an Iodinated Ethyl Linoleate

Sodium iodide (21.4 g; 142.6 mmol) is suspended in 150 ml of acetonitrile. Chlorotrimethylsilane (18.1 mL; 142.6 mmol) is added dropwise into the stirred reaction mixture, followed by addition of water (1.28 mL, 71.3 mmol). After the addition is complete, ethyl linoleate (20.0 g; 64.8 mmol) is added and the reaction stirred for 16 hrs. The reaction results in one or more of the compounds listed in Table 1.

TABLE 1
Iodinated ethyl linoleates

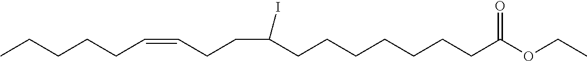 [SMILES: CCCCC/C=C\CCC(I)CCCCCCCC(=O)OCC]

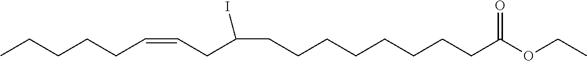 [SMILES: CCCCC/C=C\CC(I)CCCCCCCCC(=O)OCC]

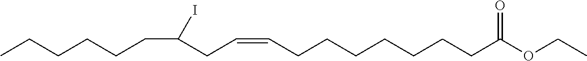 [SMILES: CCCCCCC(I)C/C=C\CCCCCCCC(=O)OCC]

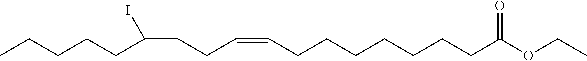 [SMILES: CCCCCC(I)CC/C=C\CCCCCCCC(=O)OCC]

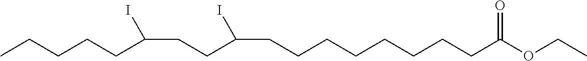 [SMILES: CCCCCC(I)CCC(I)CCCCCCCCC(=O)OCC]

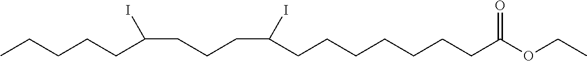 [SMILES: CCCCCC(I)CCCC(I)CCCCCCCC(=O)OCC]

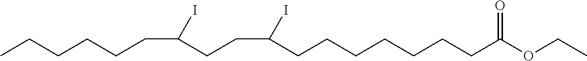 [SMILES: CCCCCCC(I)CCC(I)CCCCCCCC(=O)OCC]

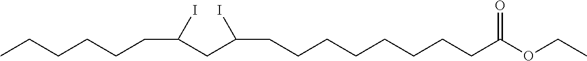 [SMILES: CCCCCCC(I)CC(I)CCCCCCCCC(=O)OCC]

The reaction is quenched by addition of water (200 mL) and extracted into diethyl ether-pentane (400 mL) and the organic phase is separated. Ether-pentane extracts are back extracted two times with 300 ml of water, one time with 100 ml of 10% aqueous 10% sodium thiosulphate and separated. The ether solution is dried over MgSO₄, filtered through a plug of basic alumina and concentrated to yield 28.2 g of product.

### Example 4: Preparation of an Iodinated Ethyl Oleate

Sodium iodide (28.9 g; 193 mmol) is suspended in 200 ml of acetonitrile. Chlorotrimethylsilane (24.5 mL; 193 mmol) is added dropwise into the stirred reaction mixture, followed by addition of water (1.73 m is, 96 mmol). After the addition is complete, ethyl oleate (50.0 g; 161 mmol) is added and the reaction is stirred for 16 hrs.

The reaction is quenched by addition of water (300 mL) and extracted into diethyl ether (300 mL) and the organic phase is separated. Ether extracts are back extracted two times with 300 ml of water, one time with 100 mL of 10% aqueous sodium thiosulphate and separated. The ether solution is dried over MgSO₄, filtered through a plug of basic alumina and concentrated to yield 61.2 g of product. The reaction results in one or more of the compounds listed in Table 2.

TABLE 2
Iodinated ethyl oleates

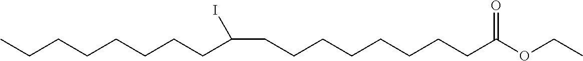 [SMILES: CCCCCCCCC(I)CCCCCCCCC(=O)OCC]

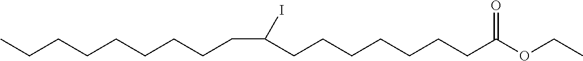 [SMILES: CCCCCCCCCC(I)CCCCCCCC(=O)OCC]

A similar procedure based on Examples 1˜4 is used to prepare iodinated methyl, propyl, isopropyl, butyl, or isobutyl esters of oleic acid, α-linolenic acid, linoleic acid, stearidonic acid, linoleladilic acid, palmitooleic acid, or arachidonic acid.

### Example 5: Preparation of Hydrolyzed Poppy Seed Oil Fatty Acids

Poppy seed oil (50 g) is mixed in a reactor with 300 mL of solution comprising of 1 M ethanolic KOH. The hydrolysis is carried out at reaction temperature 60° C. for 6 hours. After the hydrolysis, 200 mL water is added to the mixture. Unreacted materials are separated by extraction with 100 ml of hexane. The aqueous alcohol phase, containing the soaps, is acidified to pH 1 with 6N HCl, and the fatty acid mixture comprising mostly of oleic, linoleic and linolenic acid is recovered by extraction with hexane. The extract is washed with distilled water to neutral pH. The fatty acid-containing upper layer is dried over MgSO₄ and concentrated in a vacuum.

### Example 6: Preparation of Iodinated Hydrolyzed Poppy Seed Oil Fatty Acids

Sodium iodide (56.2 g; 375 mmol) is suspended in 400 ml of acetonitrile. Chlorotrimethylsilane (47.6 mL; 375 mmol) is added dropwise into stirred reaction mixture, followed by addition of water (3.37 mL, 187 mmol). After the addition is complete, hydrolyzed iodinated poppy seed oil fatty acid mixture (50.0 g; ˜150 mmol) is added and reaction is stirred for 16 hrs. The reaction is quenched by addition of water (200 mL) and extracted into diethyl ether (400 mL) and hexane (400 mL) mixture and organic phase is separated. Ether-hexane extracts are back extracted two times with 500 ml of water, one time with 250 mL of 10% aqueous sodium thiosulphate and separated. The ether-hexane solution is dried over MgSO₄, filtered and concentrated to yield 58.7 g of product.

### Example 7: Preparation of Iodinated Hydrolyzed Poppy Seed Oil Ethyl Esters

Iodinated hydrolyzed poppy seed oil (58 g, ˜135 mmol) is dissolved in anhydrous ethanol (250 mL) and 2,2-diethoxypropane (26.2 ml; 162 mmol) and treated with sulfuric acid (0.3 ml; 5.6 mmol). The mixture is heated to 65° C. for period of 16 hours and concentrated in vacuum. The mixture is re-dissolved in 200 ml of diethyl ether and 200 ml of hexane mixture, extracted two times with 250 ml of saturated sodium bicarbonate aqueous solution, one time with 250 ml of water and one time with 250 mL of brine. The ether-hexane solution is dried over MgSO₄, filtered through a plug of basic alumina and concentrated to yield 62 g of product.

### Example 8: Preparation of an Emulsion

An emulsion is prepared by mixing 2 mL of an aqueous solution of doxorubicin (20 mg/mL) with iodinated ethyl linoleate in a ratio of 1:1 to make a “water in oil” type emulsion. The 2 mL of doxorubicin solution is added to the oil and the solution is mixed between two syringes 20 times.

### Example 9: Preparation and Delivery of an Emulsion with Example 3

An emulsion is prepared by mixing 2 mL of an aqueous solution of doxorubicin (20 mg/mL) with iodinated ethyl linoleate in a ratio of 1:3 of oil to make a “water in oil” type emulsion. Initially, 1 mL of doxorubicin solution is added, and the solution mixed between two syringes 20 times. An aliquot of 0.5 mL of the doxorubicin solution is then added to the oil and once again the solution mixed between two syringes 20 times. The rest of the drug is added and mixing repeated as above.

After preparation, the emulsion is delivered using a catheter to the liver of a pig via the common hepatic artery with good radiopacity and transient stasis is observed.

### Example 10: Radiopacity Evaluation

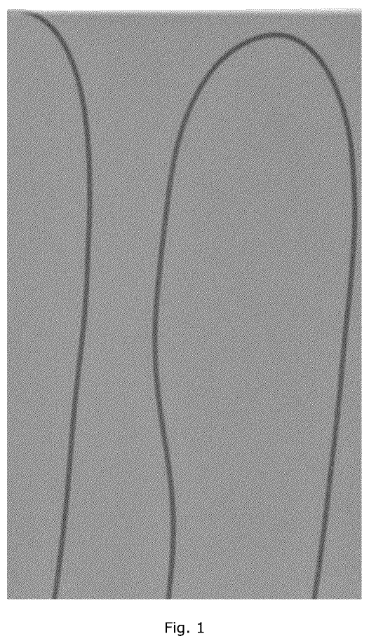

The radiopacity of iodinated ethyl linoleate (IEL) from Example 3 is compared to that of Lipiodol in a 5 Fr. Radifocus Glidecath from Terumo. Results are shown in FIG. 1 (iodized ethyl linoleate—left loop, lipiodol—right loop). Iodinated ethyl linoleate exhibits the same radiopacity as Lipiodol when loaded in a catheter.

### Example 11: HPLC Evaluation

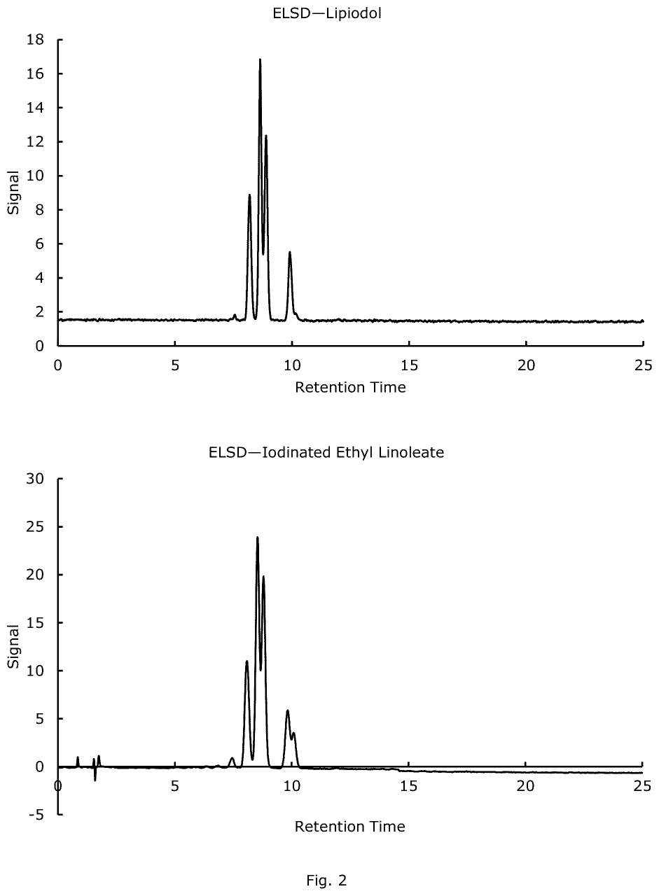

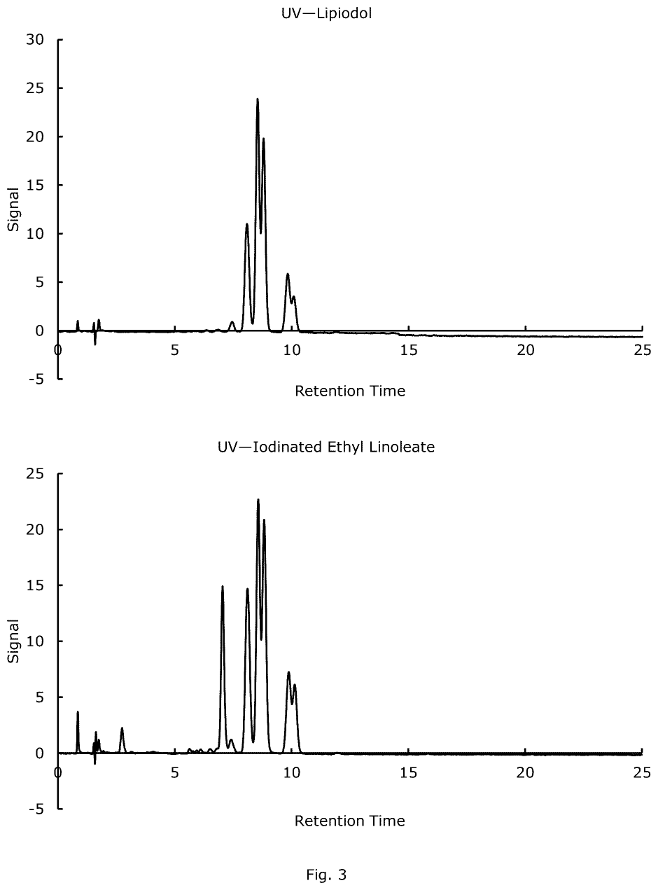

High performance liquid chromatography (HPLC) analysis of iodinated ethyl linoleate from Example 3 is compared to that of Lipiodol; evaporative light scattering detector (ELSD) and ultra-violet light detector (UV) are used. Results are shown in FIG. 2, FIG. 3, and Table 3. The HPLC test method is: Test sample concentration 0.1% in Acetonitrile; Column: ZORBAX 300SB-C18, 5 um, 4.6×150 mm; Mobile phase A: Acetonitrile; Mobile phase B: 5% acetonitrile in water; A:B=85:15; Flow rate: 1 ml/min; Column temperature: 45° C.; Injection Volume: 5 μL; UV: 210 nm; and Run time 25 min+4 min (post run).

The chemical composition of iodinated ethyl linoleate (IEL) from Example 3 evaluated by HPLC shows identical main components (in both cases, main components are iodinated isomers of ethyl linoleate). Similar composition ratio is obtained with ELSD detection for IEL and Lipiodol. Under the conditions of UV detection, the main components are also identical, the UV profile of IEL show additional minor peaks most likely representing partially iodinated ethyl linoleate.

TABLE 3
HPLC Analysis

| | | |
|---|---|---|
| Retention | 0.1% IEL in | 0.1% Lipiodol |
| Time (min.) | acetonitrile | in acetonitrile |

Signal Area-ELSD

| | | |
|---|---|---|
| 7.1 | | |
| 8.2 | 121 | 76 |
| 8.6 | 106 | 132 |
| 8.9 | 103 | 105 |
| 9.9 | 68 | 46 |

Signal Area-UV

| | | |
|---|---|---|
| 7.1 | 137 | |
| 8.2 | 199 | 150 |
| 8.5 | 251 | 268 |
| 8.8 | 244 | 229 |
| 9.8 | 101 | 86 |
| 10.0 | 80 | 45 |

### Example 12: Drug Elution

For drug elution studies, solution of the drug (Doxorubicin or Mitomycin C) at 10 mg/mL was combined with iodinated oil (Lipiodol or IEL (for example, from Example 3)) and mixed by pumping drug solution and oil back and forth in two syringes. Resulting emulsion was added into the 50 mL centrifuge tube, phosphate-buffered saline (PBS) was added, and the mixture was agitated for a specific time. Samples of PBS buffer phase were withdrawn and analyzed for drug content in specific intervals to monitor the elution.

The emulsion and elution media were prepared and combined according to Table 4.

TABLE 4

| | Emulsion | | Elution Media |
|---|---|---|---|
| | 10 mg/mL Drug Solution | Iodinated Oil | PBS |
| 1:1 | 1 mL | 1 mL | 48 mL |
| 1:4 | 1 mL | 4 mL | 45 mL |

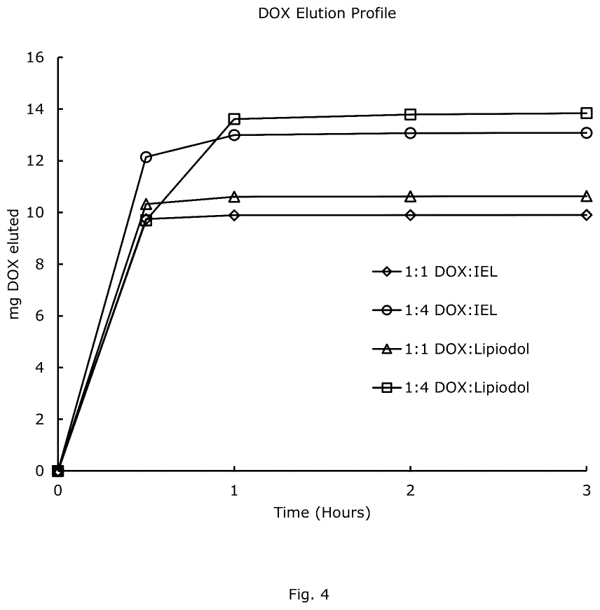

Doxorubicin elution profiles for IEL and Lipiodol (FIG. 4) show identical elution times (reaching elution plateau in 0.5-1 hour) and similar elution profiles. The elution characteristics are practically identical for both iodinated oils and the 1:1 and 1:4 emulsion ratios.

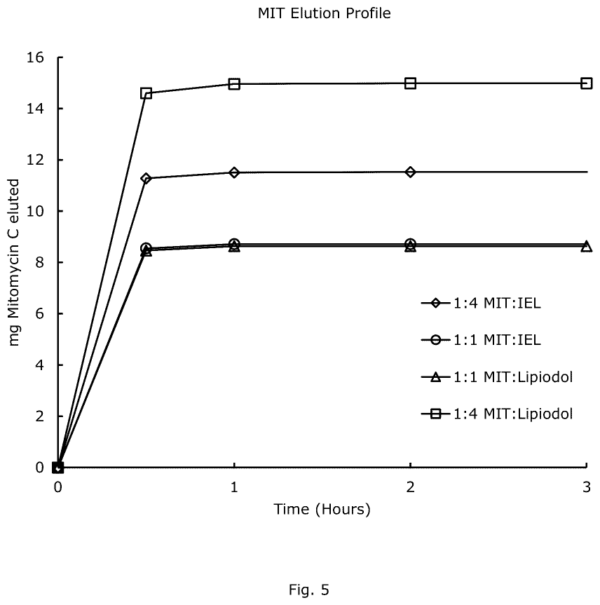

Mitomycin C elution profiles for IEL and Lipiodol (FIG. 5) show identical elution time (reaching elution plateau in 0.5-1 hour) and similar elution profiles. The elution characteristics are practically identical for both iodinated oils and the 1:1 and 1:4 emulsion ratios.

IEL shows similar characteristics to Guebert Lipiodol when evaluating physical properties (specific gravity, density, and viscosity), material composition (HPLC characterization of main components), radiopacity, and drug elution characteristics (Doxorubicin and Mitomycin C emulsion formation and elution study).

IEL has a potential for higher (and less variable) iodine content, and the production and properties are not being affected by variations related to naturally occurring raw material.

IEL preparation involve less steps and involve less reagents compared to Lipiodol preparation, hence it has a potential to yield material of superior purity.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A compound, selected from: 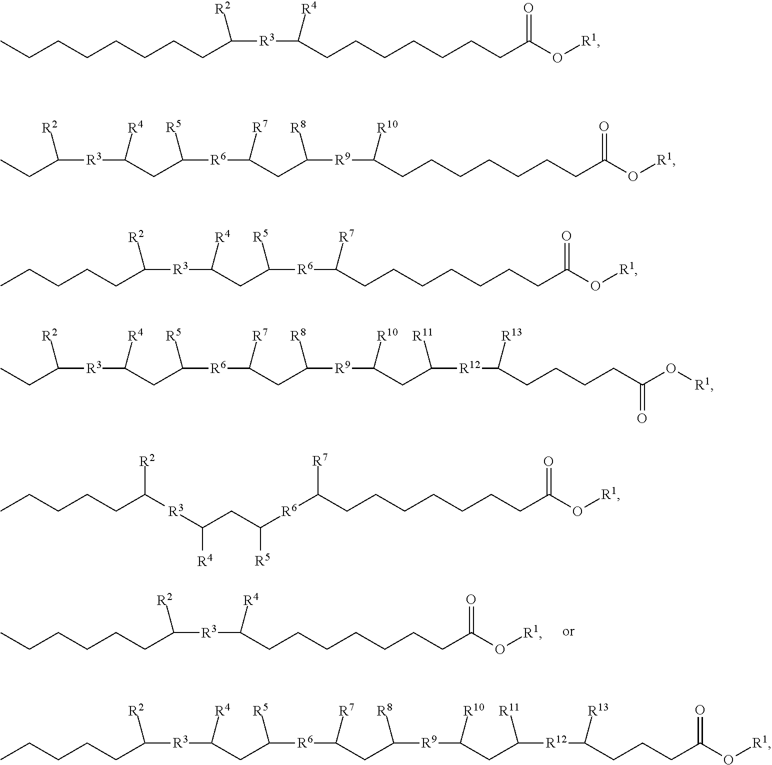
wherein
each R¹ is, independently, C₁₋₄ alkyl;
independently, R² is I, R³ is a single bond, and R⁴ is H, or R² is H, R³ is a single bond, and R⁴ is I, or R² is H, R³ is a double bond, and R⁴ is H;
independently, R⁵ is I, R⁶ is a single bond, and R⁷ is H, or R⁵ is H, R⁶ is a single bond, and R⁷ is I, or R⁵ is H, R⁶ is a double bond, and R⁷ is H;
independently, R⁸ is I, R⁹ is a single bond, and R¹⁰ is H, or R⁸ is H, R⁹ is a single bond, and R¹⁰ is I, or R⁸ is H, R⁹ is a double bond, and R¹⁰ is H; and
independently, R¹¹ is I, R¹² is a single bond, and R¹³ is H, or R¹¹ is H, R¹² is a single bond, and R¹³ is I, or R¹¹ is H, R¹² is a double bond, and R¹³ is H.

2. The compound of claim 1, selected from: 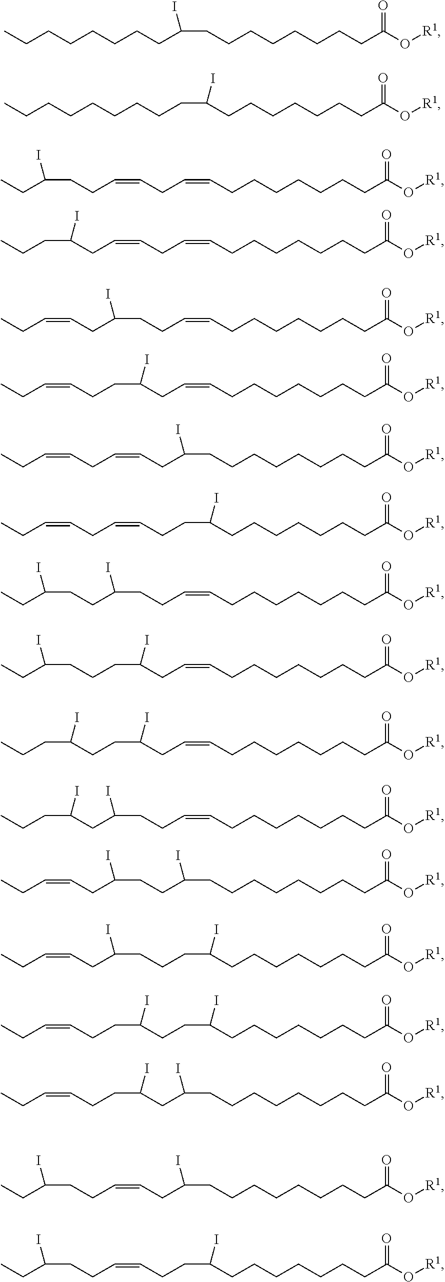 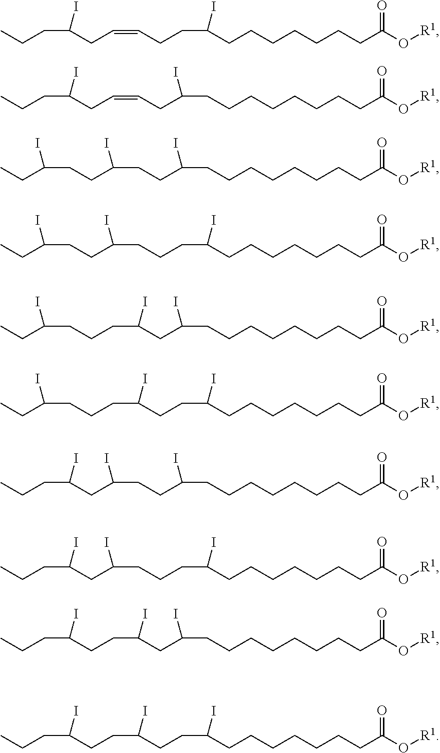 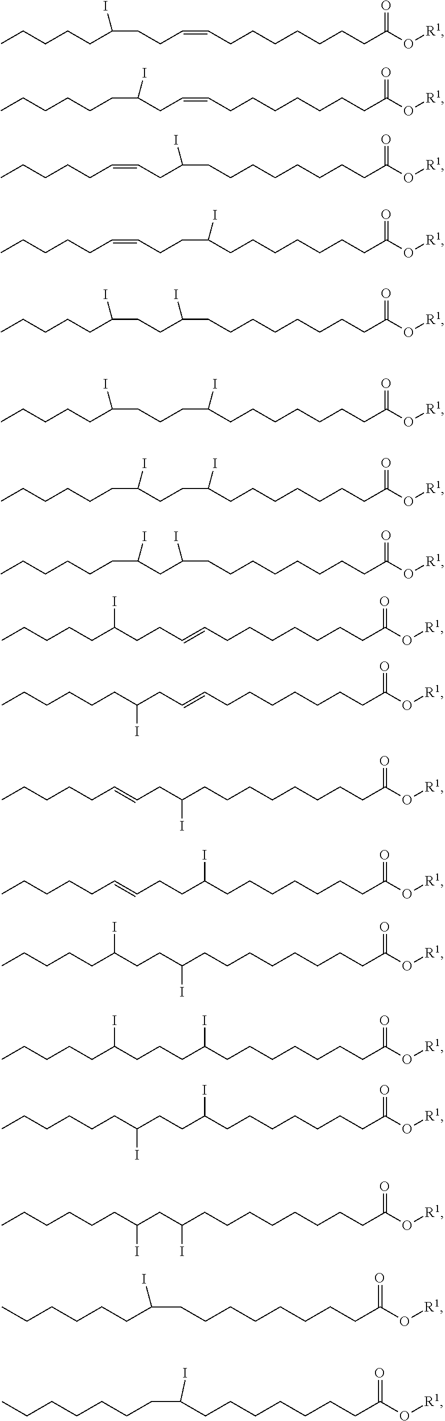
wherein
each R¹ is, independently, methyl, ethyl, propyl, isopropyl, butyl, or isobutyl.

3. The compound of claim 1, wherein
independently, R² is I, R³ is a single bond, and R⁴ is H, or R² is H, R³ is a single bond, and R⁴ is I;
independently, R⁵ is I, R⁶ is a single bond, and R⁷ is H, or R⁵ is H, R⁶ is a single bond, and R⁷ is I;
independently, R⁸ is I, R⁹ is a single bond, and R¹⁰ is H, or R⁸ is H, R⁹ is a single bond, and R¹⁰ is I; and
independently, R¹¹ is I, R¹² is a single bond, and R¹³ is H, or R¹¹ is H, R¹² is a single bond, and R¹³ is I.

4. The compound of claim 1, selected from:

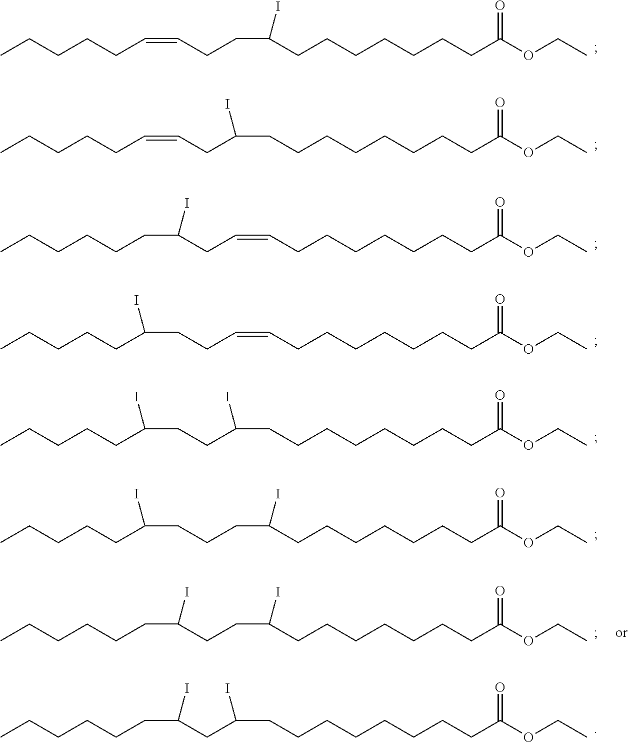

5. The compound of one of claims 1-4, prepared by a process including forming a fatty acid alkyl ester by contacting a fatty acid with a hydroxylated alkane in the presence of an acid to form the fatty acid alkyl ester, wherein the fatty acid is oleic acid, α-linolenic acid, linoleic acid, stearidonic acid, linolelaidic acid, palmitoleic acid, or arachidonic acid.

6. The compound of one of claims 1-4, prepared by a process including forming the compound by contacting a fatty acid alkyl ester with an iodide salt and a halo-silane to form the compound, wherein the fatty acid is oleic acid, α-linolenic acid, linoleic acid, stearidonic acid, linolelaidic acid, palmitoleic acid, or arachidonic acid.

7. The compound of one of claims 1-4, prepared by a process including forming an iodinated fatty acid by contacting a fatty acid with an iodide salt and a halo-silane to form the iodinated fatty acid, wherein the fatty acid is oleic acid, α-linolenic acid, linoleic acid, stearidonic acid, linolelaidic acid, palmitoleic acid, or arachidonic acid.

8. The compound of claim 7, wherein the process further includes forming the compound by contacting the iodinated fatty acid with a hydroxylated alkane in the presence of an acid to form the compound.

9. A composition, comprising the compound of one of claims 1-8.

10. A composition, comprising a purified compound of one of claims 1-8.

11. A composition, comprising at least two different compounds of one of claims 1-8.

12. A composition, comprising a plurality of the compound of one of claims 1-8, wherein the moles of alkene in the plurality of the compound is equal to or less than the moles of iodine in the plurality of the compound.

13. A composition, comprising a plurality of the compound of one of claims 1-8, wherein the moles of alkene in the plurality of the compound is at least 3 times less than the moles of iodine in the plurality of the compound.

14. A composition, comprising a plurality of the compound of one of claims 1-8, wherein the moles of alkene in the plurality of the compound is at least 10 times less than the moles of iodine in the plurality of the compound.

15. The composition of one of claims 9-14, which is a radio-opaque contrast composition.

16. The composition of one of claims 9-15, further comprising an active pharmaceutical agent.

17. A method of performing a radiologic procedure in a subject in need thereof, comprising administering the compound of one of claims 1-8 or the composition of one of claims 9-16 to the subject.

18. The method of claim 17, wherein the radiologic procedure includes hysterosalpingography.

19. The method of claim 17, wherein the radiologic procedure includes intravenous administration or intra-arterial administration.

20. The method of claim 17, wherein the radiologic procedure includes intralymphatic administration, uterine administration, fallopian administration, or intrahepatic administration.

21. A method of improving fertility in a subject in need thereof, comprising flushing a fallopian tube or uterus of the subject with the compound of one of claims 1-8 or the composition of one of claims 9-20 to the subject.

22. The method of one of claims 17-21, wherein the administering or flushing is performed via catheter.
